(19) United States
(12) Patent Application Publication
(10) Pub. No.: US 2010/0018295 A1
(43) Pub. Date: Jan. 28, 2010

(54) STATE SENSOR FOR PLANTS AND A WATERING SYSTEM COMPRISING A STATE SENSOR OF THIS TYPE

(75) Inventor: Gerd Kunze, Zwonitz (DE)

Correspondence Address: BROWDY AND NEIMARK, P.L.L.C.;624 NINTH STREET, NW, SUITE 300, WASHINGTON, DC, 20001-5303, US

(73) Assignee: RAUMEDIC AG, Munchberg (DE)

(21) Appl. No.: 12/441,495

(22) PCT Filed: Sep. 12, 2007
(86) PCT No.: PCT/EP2007/007947

§ 371 Date: Mar. 16, 2009

(30) Foreign Application Priority Data
Sep. 14, 2006 (DE) 10 2006 043 058.1

(51) Int. Cl. G01N 7/00

(52) U.S. Cl. 73/64.45; 137/561 R

(57) ABSTRACT

A state sensor for plants comprises a clamping device with two clamping elements for clamping a part of a plant. A plant parameter measuring device is coupled mechanically to the clamping device and comprises a sensor element. Said sensor element is configured as a pressure sensor element arranged on one of the clamping elements for detecting a pressure state value of the plant, which pressure state value is independent of the displacement of the clamping elements relative to one another. A watering system has at least one state sensor of this type. A reliable determination of the watering state is obtained over a long period of time and at a low cost.

The invention relates to a state or condition sensor for plants according to the preamble of claim 1 and to a watering system comprising a state sensor of this type.

Watering systems comprising plant state sensors of the type mentioned above are known from WO 02/084248 A2, JP 2002-365020 A and WO 98/33037 A1.

It is an object of the present invention to develop a plant state sensor for a watering system equipped therewith such that a reliable determination of the plant state, in particular of the watering or irrigation state, is provided over a long period of time at as low a cost as possible.

This object is achieved according to the invention by a plant state sensor having the features specified in the characterising part of claim 1.

It has been found according to the invention that a pressure state or pressure condition value of the plant, the state of which is to be monitored, is particularly well suited to the determination of the watering or irrigation state. Unlike known embodiments of plant state sensors, at least in simple embodiments of the plant state sensor according to the invention it is possible to dispense with the measurement of a plurality of plant parameters. In particular it is unnecessary to measure a leaf thickness. As recognised by the Applicant, this has the advantage that during the measurement procedure, it is possible to dispense with movable sensor components, which reduces the production cost of the sensor. The measured pressure state value of the plant is clearly correlated in particular with the watering state of said plant, so that a clear and reproducible control of a watering system with the plant state sensor is ensured by measuring the pressure state value. In addition to the watering state, the plant state sensor according to the invention is also suitable for detecting other plant states which are only correlated indirectly or are not correlated with the watering state, for example a pest attack on the plant or the electrolyte balance of the plant. To provide relative measurements of the effects of external influences, in particular of the ground and of the balance of light, a plurality of such state sensors can be spatially distributed on one or more plants and read out and the measured values of the sensors can be compared with one another.

Pressure state values according to claim 2 are particularly suitable for a measurement, since with a simple construction of the pressure sensor element, they are accessible for a direct measurement. These pressure state values are all directly correlated with the state of the plants. In the literature, the leaf pressure is also called hydrostatic excess pressure in the cell (turgor).

An arrangement of the pressure sensor element according to claim 3 results in an optimisation of the dynamic range of the watering state sensor, as the sensor element does not need to absorb all the clamping pressure exerted by the clamping device, but a predetermined amount of this clamping pressure, in particular the entire clamping pressure, is absorbed by the rigid clamping portion. In this way, the dynamic range of the pressure sensor element is optimised.

A pressure coupling layer according to claim 4 reduces undesirable measurement influences due in particular to unevennesses in the leaf surface.

A silicone pressure coupling layer according to claim 5 has an inherent elasticity well suited for use together with the pressure sensor element and is also weather-resistant. Moreover, as a result of the pressure coupling layer, the pressure sensor element can be protected in particular against the effects of the weather and against moisture.

A projecting rigid clamping portion according to claim 6, i.e. a pressure-sensitive sensor surface which springs back relative to the clamping portion allows a measuring operation in which low pressure values can be measured by the pressure sensor element. Ideally, where a freshly watered plant is concerned, the pressure measured by the pressure sensor element is zero and it rises from here as a function of the duration of a watering interval. With a surface configured thus, in particular the rigidity of the part of the plant clamped to the sensor can be measured as the pressure state value, the rigidity being directly associated with the state of the plant.

A concave surface according to claim 7 can be easily manufactured.

A projecting pressure-sensitive surface according to claim 8 entails a measured value which increases continuously with the leaf pressure and is directly correlated with the leaf pressure. This simplifies the interpretation of the measurement result.

A convex surface according to claim 9 can be produced in a cost-effective manner.

A planar and aligning surface according to claim 10 can be used to determine a water vapour pressure of the plant. This state value is directly correlated in particular with the watering state of the plant.

A flexible pressure sensor membrane according to claim 11 ensures a precise pressure measurement with an adjustable pressure measurement region. This adjustment is made by means of the pressure in the reference pressure chamber.

At least one additional sensor element according to claim 12 provides additional measuring parameters which can be used, for example to finely control the watering procedure.

A locking device according to claim 13 prevents the measured results from being undesirably influenced by a relative movement of the clamping elements with respect to one another. However, as an alternative, a pressure state value which is independent of the displacement of the clamping elements relative to one another can also be achieved in that the clamping device, independently of a displacement of the clamping elements relative to one another, clamps the part of the plant clamped between the clamping elements with a constant clamping force or with a constant clamping pressure. In so doing, the sensor element does not measure a pressure state value altered by the displacement of the clamping elements relative to one another, but under constant clamping pressure, measures a pressure state value which is dependent on the stability of the part of the plant between the clamping elements.

A UV transparent material for the clamping elements according to claim 14 prevents degradation of the part of the plant measured by the state sensor.

Ideally, the measured part of the plant is supplied in practical terms with exactly as much sunlight as the rest of the plant. UV transparent materials for the clamping elements can be: a highly UV transparent acrylic glass, for example polymethyl methacrylate (PMMA), a borosilicate glass or a high purity quartz glass.

A watering system according to claim 15 comprising the state sensor of the invention has the advantages mentioned in connection with said state sensor.

Embodiments of the invention will be described in detail in the following with reference to the drawings, in which:

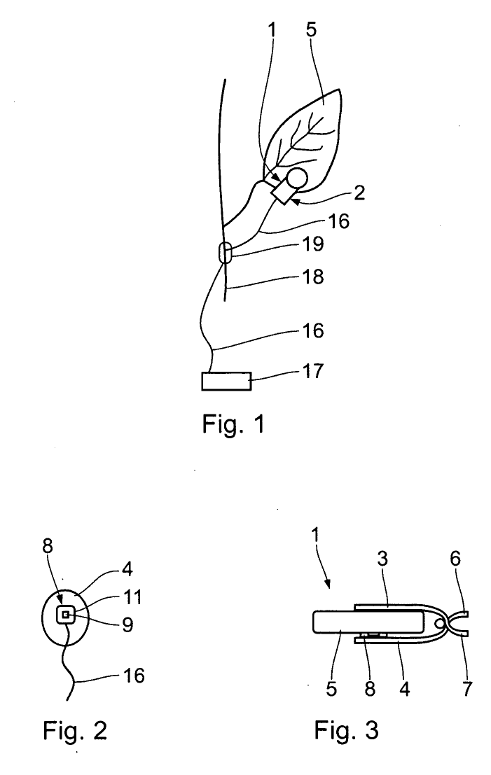

FIG. 1 schematically shows a detail of a plant with an attached state sensor using the example of a watering state sensor;

FIG. 2 shows a part of the watering state sensor of FIG. 1 with one of two clamping portions and a pressure sensor;

FIG. 3 is a side view of the watering state sensor of FIG. 1 without a supply line;

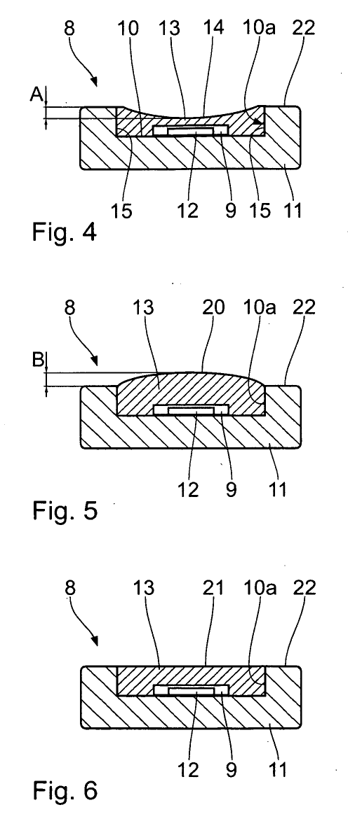

FIG. 4 is a cross-sectional view of a first variant of a pressure sensor of the watering state sensor according to FIG. 1;

FIGS. 5 and 6 show further variants of the pressure sensor;

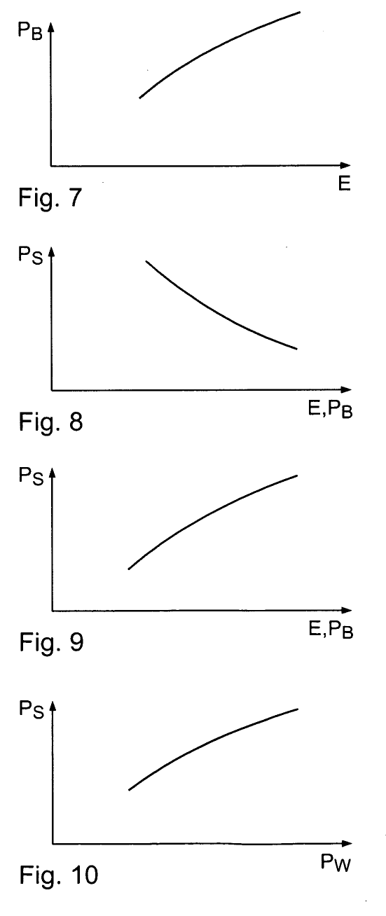

FIG. 7 schematically shows in a graph the connection between the leaf pressure P_{B} of the plant to be measured and a plant rigidity E;

FIG. 8 schematically shows in a graph the connection between the pressure sensor signal P_{S} and the rigidity E or the leaf pressure P_{B} in the embodiment of the pressure sensor according to FIG. 4;

FIG. 9 schematically shows in a graph the connection between the pressure sensor signal P_{S} and the rigidity E or the leaf pressure P_{B} in the embodiment of the pressure sensor according to FIG. 5;

FIG. 10 schematically shows in a graph the connection between the pressure sensor signal P_{S} and a water vapour pressure P_{W} of the leaf tissue of the plant to be measured in the embodiment of the pressure sensor according to FIG. 6;

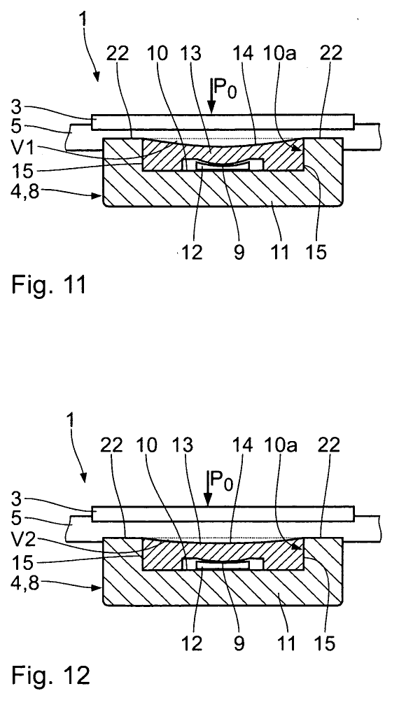

FIG. 11 is a sectional view, similar to that of FIG. 4, of the watering state sensor with the pressure sensor and a counter clamping element as well as a leaf clamped in between which has absorbed a small amount of water (leaf with dry stress);

FIG. 12 is a view, similar to that of FIG. 11, of the watering state sensor with the leaf which, compared to FIG. 11, has absorbed more water (well watered leaf); and

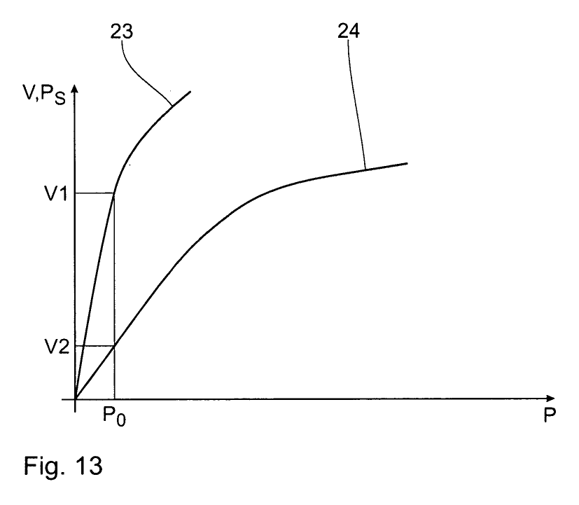

FIG. 13 is a graph which shows the dependency of a volume V pressed into a pressure coupling layer on the clamping pressure P of a clamping device of the watering state sensor with two drawn-in compressibility curves of leaf material of different watering states which is measured by the watering state sensor.

A watering state sensor 1 for plants has a clamping device 2 with two clamping elements 3, 4 for clamping part of a plant in the form of a leaf 5. A force clamping the leaf 5 between the clamping elements 3, 4 is provided by a biasing spring 6, which is supported on both clamping elements 3, 4. To grasp the leaf 5 by the clamping device 2 and to release or align the clamping device 2 relative to the leaf 5, the clamping elements 3, 4 can be released by means of a gripping and actuating unit 7 positioned on the other side of the biasing spring 6. The clamping device 2 can have a locking unit (not shown) which prevents the clamping elements 3, 4 from moving away from each other after grasping and aligning the leaf 5. The clamping elements 3, 4 can be made of a UV transparent material such that where the clamping device 2 covers the leaf 1, photosynthesis can also take place in the leaf 5. Examples of materials for the UV transparent material of the clamping elements 3, 4 are a highly UV transparent acrylic glass, for example polymethyl methacrylate (PMMA), a borosilicate glass or a high purity quartz glass.

Arranged between one of the clamping elements, namely the clamping element 4 shown below in FIG. 3 and the leaf 5 is a plant parameter measuring device in the form of a pressure sensor 8 which is rigidly connected to the clamping element 4. Therefore, in the following, the clamping element 4 will also be called a pressure sensor clamping element. The pressure sensor 8 is coupled mechanically to the clamping device 2 by this arrangement.

In a first embodiment of the pressure sensor 8 according to FIG. 4, said pressure sensor 8 has a pressure sensor membrane 9 as a sensor element. The pressure sensor membrane 9 is positioned on a base 10 of an upwardly open recess 10a in FIGS. 3 and 4 of a rigid sensor housing 11 made of metal or ceramics. An embodiment of the sensor housing 11 made from a plastics material, for example PMMA (polymethyl methacrylate) or PEK (polyethyletherketone) is also possible. In particular, the sensor housing 11 can be made from titanium. Since the leaf 5, as shown in FIG. 3, is clamped between the upper clamping element 3 in FIG. 3 and the sensor housing 11, the sensor housing 11 is simultaneously a clamping portion of the pressure sensor clamping element 4.

To give a pressure measurement range of the pressure sensor 8, the pressure sensor membrane 9 is connected to a reference pressure channel 12 arranged on the side of the pressure sensor membrane 9 remote from the leaf.

The pressure sensor membrane 9 is embedded in a resilient pressure coupling layer 13 made of silicone. Said pressure coupling layer 13 has, towards the leaf 5, a recessed, in particular concave measuring window surface 14, such that the pressure coupling layer 13 does not project over the edge-side boundary of the recess 10a in the sensor housing 11, but springs back by a distance A with respect to this edge-side boundary in the measuring region of the pressure sensor membrane 9. In the region of side walls 15, the pressure coupling layer 13 is flush with the edge of the sensor housing 11 around the recess 10a.

The pressure sensor 8 is connected to a schematically shown readout device 17 via a supply line 16. The supply line 16 is secured between the pressure sensor 8 and the readout device 17 on a more stable part of the plant compared to the leaf 5, namely a branch 18, by means of a fixing element 19. The fixing element 19 can be a further clamp.

The watering state sensor 1 is positioned and used to determine the watering state of a plant as follows: first of all, the pressure sensor 8 is firmly clamped to the leaf 5 using the clamping device 2 so that the side of the sensor housing 11 facing the leaf is pressed with a predetermined pressure against the tissue of the leaf 5. The edge of the sensor housing 11 surrounding the recess 10a is configured to be wide enough to prevent disturbing force vectors. The leaf 5 is clamped in the clamping device 2 in a predetermined watering state, for example a predetermined time after the regular watering. After clamping by the clamping device 2, it is ensured that the clamping elements 3, 4 do not deviate outwards by a displacement in respect of a change in the pressure exerted by the leaf 5 on the clamping elements 3, 4, hereinafter also called the leaf pressure P_{B}. This securing measure can be performed using the aforementioned locking device.

The leaf pressure P_{B} is an indication of the watering state of the plant. The higher the leaf pressure P_{B}, the more water the leaf 5 has absorbed at the time of the measurement. Renewed watering is necessary when the leaf pressure P_{B} falls below a predetermined limiting value. Correlated with the leaf pressure P_{B} is a leaf rigidity value E which, in turn, is associated with the modulus of elasticity of the plant. The rigidity E is a function of the characteristics, dependent on the watering state, of the cell walls of the plant. FIG. 7 illustrates the correlation of the leaf pressure P_{B} with the rigidity. As the rigidity E increases, so does the leaf pressure P_{B}.

FIG. 8 shows by way of example and very schematically the dependence of the measured value of the pressure sensor 8, P_{S}, on the rigidity E or the leaf pressure P_{B}. In the case of a high rigidity E and a high leaf pressure P_{B}, i.e. a good watering state of the plant, the plant tissue is so rigid that it bridges the depression in the measuring window surface 14 in the recess without the leaf 5 resting on the measuring window surface 14. In this limiting case, the pressure sensor 8 does not measure any contact of the pressure sensor membrane 9, i.e. no pressure exerted by the leaf 5 (P_{S}=0). When the watering state deteriorates, the leaf pressure P_{B} and also the rigidity E fall, so that the leaf 5 is pressed by the clamping device 2 into the depression of the recess 10a and presses against the pressure sensor membrane 9 via the measuring window surface. As the leaf pressure P_{B} or rigidity E drops, the pressure sensor 8 thus measures a rising sensor pressure P_{S}, as shown in FIG. 8. As soon as the measured pressure value P_{S} exceeds a predetermined limiting value, a control unit of the readout device 17 activates a watering mechanism for the plant, so that the plant is watered until the measured value P_{S} is again below a second, lower limiting value, thus until the leaf pressure P_{B} or the rigidity E has again exceeded a predetermined measurement, due to watering. In this manner, the watering state of the plant can be maintained at a predetermined level.

FIG. 5 shows a further embodiment of a pressure sensor 8. Components corresponding to those which have already been described above while bearing in mind the embodiment of the pressure sensor according to FIG. 4, have been given the same reference numerals and will not be discussed again in detail. The pressure sensor 8 according to FIG. 5 differs from that of FIG. 4 in that a measuring window surface 20 of the pressure sensor 8 according to FIG. 5 is configured to project over the recess 10a by a distance B. In the embodiment shown, this projection is convex, i.e. is at its highest in the centre above the pressure sensor membrane 9.

The pressure sensor 8 according to FIG. 5 is used as follows for measuring the watering state of the plant with the leaf 5: after clamping, aligning and optionally locking the clamping device 2 of the pressure sensor 8 according to FIG. 5 in a predetermined watering state of the plant, the pressure sensor 8 indicates a measured value P_{S} which corresponds to the total of the clamping pressure of the clamping device 2 and of the leaf pressure P_{B} Values of the leaf pressure P_{S} in a range between 50 and 150 mmHg are produced. As the leaf pressure P_{B} or rigidity E falls, so the measured pressure P_{S} also falls, as shown in FIG. 9. Thus, as already explained above, below a predetermined first pressure limiting value, the watering of the plant with the leaf 5 is activated until a higher second pressure limiting value is reached once again.

FIG. 6 shows a further embodiment of a pressure sensor 8 of a watering state sensor 1. Components of the pressure sensor 8 corresponding to those which have already been described above with reference to FIGS. 4 and 5 have been given the same reference numerals and will not be discussed again in detail.

In the case of the pressure sensor 8 according to FIG. 6, a measuring window surface 21 aligns over the entire recess 10a with a peripheral surface 22 of the sensor housing 11 which surrounds the recess 10a. In the embodiment according to FIG. 6, the peripheral surface 22 which is also present in the embodiments according to FIGS. 4 and 5 as a clamping portion surrounding the pressure sensor membrane 9 is made of a material which seals the sensor housing 11 around the recess 10a against the resting leaf 5. The vapour pressure which develops above the surface of the leaf 5 cannot escape out of the gap between the leaf 5 and the pressure coupling layer 13 due to this sealing effect. Thus, the measured pressure value P_{S} of the pressure sensor 8 according to FIG. 6 is an indication of the water vapour pressure P_{W} of the leaf 5, as shown in FIG. 10.

The pressure sensor 8 according to FIG. 6 is used as follows: after clamping, aligning and optionally locking the clamping device, the pressure sensor 8 according to FIG. 6 indicates a measured value P_{S} which corresponds to a first water vapour pressure. If the plant is subsequently not watered, the water vapour pressure P_{W} and thus the measured pressure P_{S} drops, as shown in FIG. 10. As soon as the measured value P_{S} falls below a first predetermined limiting value, the readout device 17 activates the watering of the plant with the leaf 5 until the measured value P_{S} has risen to a higher second predetermined value.

FIGS. 11 to 13 show more clearly in detail the function, already described above, of the watering state sensor 1, said watering state sensor 1 not having a locking device in the embodiments according to FIGS. 11 and 12.

FIG. 11 shows the situation where a leaf 5 has not been sufficiently watered. Due to the fact that the leaf 5 has not been sufficiently watered, said leaf 5 is limp and can be easily compressed. The clamping pressure P exerted by the clamping element 3 on the pressure sensor 8 which is simultaneously the counter clamping element 4, means that between the clamping element 3 and the peripheral surface 22, the limp leaf 5 is compressed to a fraction of its actual thickness. Thus, a relatively large amount of leaf material, namely in total a volume V1, presses onto the pressure coupling layer 13 above the pressure sensor membrane 9. Since the pressure coupling layer 13 is incompressible, the pressure sensor membrane 9 is pressed downwards. The leaf volume V1 in the situation “leaf with dry stress” according to FIG. 11 above the pressure coupling layer 13 pressing on said layer 13 thus results, with a clamping pressure P₀, in a relatively high sensor measured value P_{S} which is directly correlated with the value of the pressed-in volume V1. This is illustrated in the graph according to FIG. 13, the initially steeply inclined compressibility curve 23 belonging to the leaf with dry stress.

FIG. 12 shows the situation of a well watered leaf 5. The same clamping pressure P₀ as in the situation according to FIG. 11 results in a lower compression of the leaf 5 between the clamping element 3 and the peripheral surfaces 22 due to the higher rigidity E of the leaf 5. This means that the distance of the clamping element 3 from the peripheral surfaces 22 in the “well watered leaf” situation according to FIG. 12 is greater than in the situation according to FIG. 11. In the “well watered leaf” situation, the clamping pressure P₀ of the clamping element 3 presses a smaller leaf volume V2 compared to the situation of FIG. 11 towards the measuring window surface 14 and the pressure sensor membrane 9. Said pressure sensor membrane 9 is thus deflected outwards to a lesser extent, which results in a lower measured value P_{S} of the pressure sensor 8. This situation is illustrated in the graph of FIG. 13 as a less steeply inclined compressibility curve 24.

In FIGS. 11 and 12, the volume displacements V1, V2 and the corresponding deflections of the pressure sensor membrane 9 are not drawn to scale, but are exaggerated.

It can be inferred from the comparison of the two curves 23, 24 of FIG. 13 that in the region of the clamping pressure P₀ mentioned by way of example in connection with FIGS. 11 and 12, higher or lower clamping pressures P also prevail, for which there exists a dependence, which can be evaluated for the measurement of the plant state, of the volume V and thus of the sensor measured value P_{S}, on the leaf state. The curves 23, 24 are an indication of the compressibility of the leaf 5, i.e. of the reciprocal of the modulus of elasticity or of the rigidity E.

In the embodiment according to FIGS. 4, 11 and 12, the peripheral surface 22 and the surface of the clamping element 3 facing said peripheral surface 22 are planar and run parallel to one another. It is also possible to configure these mutually facing surfaces in a complementary manner to one another, for example undulating in a convex-concave or concave-convex manner or in a complementary manner with respect to one another. This can be advantageous in respect of fixing the leaf and with regard to concentrating the leaf volume displacement towards the pressure sensor membrane 9.

In addition to the pressure sensor, the watering state sensor 1 can also comprise further sensors for determining at least one of the following parameters: temperature, incidence of light, atmospheric moisture.

The clamping element 3 is either made completely of a biologically compatible material, or where it rests against the leaf 5, is coated in a biologically compatible manner. The same applies accordingly to the sensor housing 11. The clamping element 3 is made in particular of the same material as the sensor housing 11.

With a watering state sensor according to the invention, the watering state of the plant having the leaf 5 can be determined over a long period of time, for example over several days or weeks. Other plant states can also be measured with the state sensor 1. Thus, it is possible to determine whether a plant has been attacked by a pest. The electrolyte balance of the plant can also be monitored. A plurality of state sensors 1 can be positioned distributed on one or more plants to perform the relative measurements, in order to ascertain the degree of external influences, for example of the ground or light balance, on individual plants or parts of plants.

1. A state sensor (1) for plants
comprising a clamping device (2) with two clamping elements (3, 4) for clamping a part (5) of a plant,
comprising a plant parameter measuring device (8) coupled mechanically to the clamping device (2), with a sensor element (9),
wherein the sensor element (9) is configured as a pressure sensor element which is arranged on at least one of the clamping elements (4) and is designed to detect a pressure state value (P_{B}, E, P_{W}), independent of a displacement of the clamping elements (3, 4) relative to one another, of the plant.

2. A state sensor (1) according to claim 1, comprising a configuration of the sensor element (9) such that a pressure of a part of a plant is detected as the pressure state value.

3. A state sensor according to claim 1, wherein the pressure sensor clamping element (4), on which the sensor element (9) is arranged comprises a rigid clamping portion (11) surrounding the sensor element (9), the sensor element (9) being accommodated in a clamping-side recess (10a) in the clamping portion (11).

4. A state sensor according to claim 1, wherein the sensor element (9) is embedded in a resilient pressure coupling layer (13).

5. A state sensor according to claim 4, comprising a pressure coupling layer (13) made of silicone.

6. A state sensor according to claim 1, wherein a pressure-sensitive surface (14) of one of the group of the sensor element (9) and the pressure coupling layer (13) is configured such that the rigid clamping portion (11) projects over the pressure-sensitive surface of the sensor element (9).

7. A state sensor according to claim 6, wherein the pressure-sensitive surface (14) of one of the group of the sensor element (9) and of the pressure coupling layer (13) is concave.

8. A state sensor according to claim 1, wherein a pressure-sensitive surface (20) of one of the group of the sensor element (9) and of the pressure coupling layer (13) is configured such that the pressure-sensitive surface (20) projects over the rigid clamping portion (11).

9. A state sensor according to claim 8, wherein the pressure-sensitive surface (20) of one of the group of the sensor element (9) and of the pressure coupling layer (13) is convex.

10. A state sensor according to claim 1, wherein a pressure-sensitive surface (21) of one of the group of the sensor element (9) and of the pressure coupling layer (13) is planar and aligns with the rigid clamping portion (11).

11. A state sensor according to claim 1, wherein the sensor element (9) is configured as a flexible pressure sensor membrane which is in contact with a reference pressure chamber (12).

12. A state sensor according to claim 1, comprising at least one additional sensor element for at least one of the following parameters: temperature, incidence of light, atmospheric moisture.

13. A state sensor according to claim 1, comprising a locking device for providing a fixed relative position of the clamping elements (3, 4) with respect to one another after the part (5) of the plant has been clamped.

14. A state sensor according to claim 1, wherein at least one of the clamping elements (3, 4) is made of a UV transparent material.

15. A watering system comprising at least one state sensor according to claim 1 and an evaluation device (17) which is in a signal connection (16) with the pressure sensor element (9).

16. A state sensor (1) according to claim 1, comprising a configuration of the sensor element (9) such that one of the group of a lead pressure (P_{B}) and a plant part rigidity (E) and a plant water vapour pressure (P_{W}) is detected as the pressure state value.
